(19) United States
(12) Patent Application Publication
(10) Pub. No.: US 2008/0254115 A1
(43) Pub. Date: Oct. 16, 2008

(54) Micropellet Containing Pellets and Method of Preparing Such Pellets

(76) Inventor: Orapin P. Rubino, Towaco, NJ (US)

Correspondence Address: HEDMAN & COSTIGAN P.C., 1185 AVENUE OF THE AMERICAS, NEW YORK, NY, 10036, US

(21) Appl. No.: 11/596,090

(22) PCT Filed: May 13, 2005
(86) PCT No.: PCT/US2005/016981

§ 371 Date: Nov. 9, 2006

Related U.S. Application Data
(60) Provisional application No. 60/572,599, filed on May 19, 2004.

(51) Int. Cl. A61K 9/14; A61K 47/38; A61K 47/30; A61K 47/02; A61K 9/48

(52) U.S. Cl. 424/456; 424/489; 514/781; 514/769; 514/770; 514/772.5; 514/772.6; 424/490

(57) ABSTRACT

The invention provides novel pellets adapted for biologically active preparations and a novel process for preparing said pellets. The novel pellets are adapted for use in the delivery of a biologically active agent. The pellets have an inner zone comprising a plurality of micropellets which are bound together to form a pellet when the micropellets are dispersed in a matrix of an inert pharmaceutical excipient, a biologically active agent and optionally having an outer zone comprising a surface layer comprising a pharmaceutical excipient with or without a biologically active agent. The pellets will have an arcuate surface due to the manner in which they are formed.

### BACKGROUND OF THE INVENTION

Oral solid dosage forms for biologically active agents have been prepared using various techniques that have been used to combine a powdered biologically active agent substance with a diluent and to form that mixture into a physical form that is suitable to make powder filled capsules, compressible particles for making tablets or coatable particles that are adapted for controlled release of active substances using matrix forming additives or membrane based controlled release coatings. As used herein, the term “biologically active agent” is used to include pharmaceutical compounds, pharmaceutical compositions, vitamins and nutrients.

The prior art has used various wet granulation, dry granulation, fluidized-bed, extrusion-spheronization and direct compression techniques to prepare particles in the form of granules or pellets for making solid dosage forms. In addition, spray-drying and spray congealing techniques have been used to form these types of particles.

The use of fluidized beds has been based on the use of top-spray or bottom-spray techniques using a Wurster air suspension column or a tangential-spray in rotary fluid-bed coater/granulator. Apparatus which have been used for coating and/or making pellets are described in U.S. Pat. No. 4,895,733; U.S. Pat. No. 5,132,142 and U.S. Pat. No. 6,354,728 all of which are incorporated by reference. South African patent 20000169 describes certain pharmaceutical pelleted formulations which contain up to 90 wt. % of a pharmaceutically active ingredient which are made by conventional spheronization techniques.

As used herein the term “pellet” means a substantially spherically shaped particle having a aspect ratio (a ratio of the length of the pellet divided by the width found at an angle of 90° in respect to the length) which is less than about 1.4, more preferably less than about 1.3, even more preferably less than about 1.2, especially preferably less than about 1.1, and most preferably less than about 1.05 and an approximate average diameter of 0.25 to 2.5 mm.

As used herein the term “micropellet” means a shaped particle which may have an irregular shape, a spherical shape or a cubic shape having a aspect ratio (a ratio of the length of the pellet divided by the width found at an angle of 90° in respect to the length) which is less than about 1.4, more preferably less than about 1.3, even more preferably less than about 1.2, especially preferably less than about 1.1, and most preferably less than about 1.05 and an approximate average diameter of 50 to 500 microns, preferably 50 to 200 microns.

The micropellets may comprise a biologically active agent with an osmotic agent and/or an inert pharmaceutical excipient. The micropellets are provided with a coating of a pharmaceutically acceptable water insoluble polymer using a conventional coating technique such as a technique which employs a Wurster coating apparatus. The coating thickness typically employed will be a sufficient amount of coating material which will prevent the micropellets from losing their structural integrity during processing to form pellets. A coating which has a thickness of from 1-10 microns and preferably is from 3-7 microns will be sufficient for this purpose. The term “water insoluble binder” is used herein to mean a pharmaceutically acceptable, polymeric coating material which is insoluble in water or a polymer which when placed in contact with a 50:50 mixture of water and polymer at ambient conditions, will not dissolve more than 50% of the total amount of polymer in one hour.

In one aspect, the present invention comprises the use of a rotating device that propels the powder particles onto a tangentially arranged surface which causes the powder particles to roll on said tangentially arranged surface. This process results in pellets having a controlled density, for instance highly dense pellets. These pellets may be formulated to have matrix controlled release properties or other types of release properties depending on the pharmaceutical excipients which are employed. The pellets may be: adapted to contain high levels of biologically active agents, i.e. more than 90 wt %, such as more than 95 wt % and in particular more than 99 wt % and even more than 99.9 wt % of a biologically active agent in each pellet; pellets that are directly manufactured with a narrow size distribution without the need to carry out any substantial separation step and pellets that have multiple biologically active agent and/or rate release controlling coatings which will provide for controlled release of the active agents and/or physical separation of incompatible agents that are advantageously administered in combination. The pellet may comprise sustained release, pulsatile release, enteric release, immediate release or a combination of these release characteristics. In addition, the present invention provides novel processing methods which can optionally be used to reduce or eliminate the use of organic solvents, can produce smaller particles, can reduce the number of process steps and increase the total throughput per operating unit due to greatly reduced processing cycles.

### SUMMARY OF THE INVENTION

The invention provides novel pellets adapted for biologically active preparations and a novel process for preparing said pellets. The novel pellets are adapted for use in the delivery of a biologically active agent. The pellets have an inner zone comprising a plurality of micropellets which are bound together to form a pellet when the micropellets are dispersed in a matrix of a inert pharmaceutical excipient, a biologically active agent and optionally having an outer zone comprising a surface layer comprising a pharmaceutical excipient with or without a biologically active agent. The pellets will have an arcuate surface due to the manner in which they are formed.

The process of the invention comprises feeding micropellets into a device suitable for contacting and adhering said micropellets. According to one embodiment, the process may be started by feeding micropellets. In this case, pellet cores are formed from said micropellets. Micropellets are brought into contact such that some of the contacts lead to an adherence of micropellets to one another with a pharmaceutical excipient or a binder. It is usually preferred to use a pharmaceutically acceptable liquid in conjunction with the initial step of forming micropellets into a pellet.

The micropellets may be aggregated by spraying a binder solution in the apparatus disclosed in U.S. Pat. No. 6,354,728. Alternatively, an aqueous pharmaceutically acceptable diluent, e.g. water, may be sprayed onto the micropellets with the simultaneous addition of dry powder to form the micropellets into pellets.

The process of the present invention may be carried out in a rotating device that propels the micropellets onto a tangentially arranged surface which causes the micropellets to roll on said tangentially arranged surface and adhere to other micropellets thus forming pellets as the particles roll on the tangential surface. The rolling movement on the tangential surface is believed to result in a compacting force which is exerted on the adhering micropellets during the rolling movement.

A preferred device comprises a rotor and a chamber wherein said rotor is located. On rotation of said rotor, the pellets being formed move in an outward direction on said rotor. Ultimately, the pellets come into contact with an inner wall of said chamber which is arranged to receive the outwardly moving pellets tangentially so that the pellets will begin to roll as they contact the inner wall of the chamber.

The preferred device also contains mechanical guide means arranged above said rotor such that the pellets being formed, after leaving said rotor, are guided back onto said rotor. Thus, the pellets being formed are put into circulation within the device. This allows the pellets being formed to repeatedly come into contact with micropellets fed with a pharmaceutically acceptable liquid and optionally a binder. Thereby micropellets may adhere and grow into larger pellets. The adhering powder micropellets are then formed into pellets as the micropellets undergo a rolling movement, e.g. on one of the surfaces of the device including the guide means. An especially preferred device for carrying out the process of the invention is disclosed in U.S. Pat. No. 6,354,728. The use of this device offers the advantage of a particularly effective rolling movement of pellets in a concussion free manner. In this way, damaging the pellets being formed can be avoided. On the other hand, an effective uptake of energy can be achieved.

In addition to rolling on surfaces of the device in which the process is carried out, such as on the rotor surface, the inner wall of the chamber and the surface of the mechanical guide means, the rolling movement also involves rolling interactions within the bed of pellets being formed. These interactions are based on the spin of the pellets being formed. During the rolling movement of the pellets being formed on surfaces of the device used for carrying out the process, the pellets acquire a spin. A pellet being formed which rolls on surfaces of the device will transfer part of its spin to pellets in direct contact with it. Thus, even pellets which are, during a particular phase of the process, not in direct contact with a surface of the device, will perform a rolling movement, more precisely a rolling movement relative to other pellets, contributing to the formation of the pellets.

Thus, it is preferable to carry out the process in such a manner that at least during a part of the processing time an individual pellet being formed comes into intimate contact with other pellets being formed. This requires the quantity of pellets processed in one batch to be sized to provide a sufficient number of intimate contacts with other micropellets in order to cause the final pellets to have the desired properties. Generally, the apparatus that is used in the practice of the invention should be operated with an initial load of 25 to 100% of the volume capacity of the rotor. In any event, the apparatus should be operated with a sufficient load of micropellets that individual micropellets are continuously contacted with other micropellets.

If the rotation of a rotor is used to supply kinetic energy to the pellets being formed, the energy supply can be varied by varying the rotor speed. The rotor speed is a process parameter that can be varied to modify the size of the pellets that are formed from the micropellets.

The selected rotor speed will impart a radial velocity to the micropellets/pellets which has been found to affect the formation of the final pellet. Generally, it has been found that rotor speed that impart a radial velocity (measured at the tip of the rotor) of about 3-10 meters/second, will in the case of most biologically active materials, produce a pellet which is an aggregate of micropellets and at a higher radial velocity the micropellets are not readily formed into aggregates.

The spray rate and powder feed rate may be varied to control the size of the pellets and the rate at which the pellets are formed.

As disclosed herein, the invention contemplates feeding, a portion of the pharmaceutical excipient powder used to make the pellet, in the form of a dry powder as the final or terminal step in the formation of the pellets. A terminal step of feeding the dry powder may be used to improve the smoothness of the surface of the pellets.

Release rates may be determined in a USP 23, Type II dissolution apparatus using water as a dissolution media. at 37° C. at a stirring speed of 100 rpm.

An apparatus suitable for carrying out this embodiment of the process of the invention is disclosed in U.S. Pat. No. 6,354,728. This device comprises a rotor located in a chamber such that an annular gap exists between the rotor and the inner wall of said chamber. Alternatively or in addition, the rotor may contain openings in its surface allowing a gas to pass through.

The gas stream, through the openings in the rotor, may be directed such that forces acting on the pellets being formed are reduced or increased. For instance, a gas may be led through openings in the rotor from below to reduce interactions between pellets and the rotor surface as well as among the pellets. In a preferred embodiment, the invention provides a spherically shaped pharmaceutical pellet, comprising micropellets in a matrix optionally having at least one or more layers surrounding, the matrix The layers may be formed from a powder adhering to the matrix or from a post pelletization coating.

Preferably, the pellets are formed from a matrix containing micropellets dispersed in a powder comprising a biologically active agent and a pharmaceutical excipient or excipients and in certain embodiments may have two or more outer layers superimposed on the pellets which adhere to one another.

Generally the micropellets according to the invention will have an average diameter of from 50 to 500 microns or preferably from 50 to 200 microns. The layer or layers on the pellets will preferably be from 1-10% of the total thickness of the pellet and more preferably from 1 to 5% of the thickness of the pellet. The pellets, of a specific composition, prepared according to the invention preferably have a narrow particle size distribution such that a maximum of 20% by weight of the pellets have a diameter deviating from the average diameter of all by more than 20%. Preferably, a maximum of 10% by weight of the pellets have a diameter deviating from the average diameter of all, by more than 20%. Further preferably, a maximum of 20% by weight of the pellets have a diameter deviating from the average diameter of all pellets by more than 10% by weight. An especially preferred micropellet product has a particle size distribution such that a maximum of 10% by weight of the pellets have a diameter deviating from the average diameter of all pellets by more than 10% by weight. All percents by weight are based on the total weight of the pellets.

Generally the pellets according to the invention will have an average diameter of from 0.25 mm to 2.5 mm, and preferably from 0.70 mm to 1.5 mm. All percents by weight are based on the total weight of the pellets.

If desired, the pellets may be made from micropellets which have an irregular shape, a cubic shape or a substantially spherical shape.

The invention also provides a process for making pharmaceutical pellets as described herein wherein the pellets are formed by (a) contacting micropellets, adhering them to each other and compacting said adhered micropellets by a rolling movement and (b) feeding a sufficient amount of a composition comprising a pharmaceutical excipient alone or in combination with a biologically active agent to form said micropellets into a matrix optionally having an outer zone comprising a layer formed from either an excipient alone or in combination with a biologically active agent which are the same or different from the biologically active agent and/or an excipient used to form the matrix.

A optional embodiment of the invention provides a process of preparing pellets by:

(a) forming micropellets which comprise a water insoluble polymer as a coating and a biologically active agent, with or without an osmotic agent;
(b) feeding said micropellets to an operating apparatus which comprises a rotor chamber having an axially extending cylindrical wall, means for passing air through said chamber from the bottom, spray means for feeding a liquid into said chamber, a rotor which rotates on a vertical rotor axis, said rotor being mounted in said rotor chamber, said rotor having a central horizontal surface and, in at least the radial outer third of said rotor, the shape of a conical shell with an outward and upward inclination of between 10° and 80°, said conical shell having a circularly shaped upper edge which lies in a plane which is perpendicular to the rotor axis, feed ports for introducing a powdered excipient, a plurality of guide vanes having an outer end affixed statically to said cylindrical wall of said rotor chamber above a plane formed by the upper edge of said conical shell of said rotor and an inner end which extends into said rotor chamber and is affixed tangentially to said cylindrical wall of said rotor chamber and having, in cross-section to the rotor axis, essentially the shape of an arc of a circle or a spiral, such that said powdered product which is circulated by kinetic energy by said rotor under the influence of kinetic energy, moves from said rotor to an inside surface of said guide vanes before falling back onto said rotor;
(c) rotating said rotor, while feeding air and spraying a pharmaceutically acceptable liquid into said rotor chamber for a sufficient amount of time to form solid pellets having a desired diameter.

Optionally, a sufficient amount of a pharmaceutical excipient with or without a biologically active agent may be fed to the apparatus to provide said pellets with an outer zone comprising a layer comprising a pharmaceutical excipient with or without a biologically active agent.

Accordingly, it is a primary object of the present invention to provide novel pellets which are useful for the delivery of biologically active agents.

It is also an object of the invention to provide novel pellets which can contain more than 90 wt % of an active biological agent, such as a pharmaceutical.

It is also an object of the invention to provide pellets which have matrix release characteristics.

These and other objects of the invention will become apparent from the appended specification.

### BRIEF DESCRIPTION OF THE DRAWINGS

FIG. 1 is a scanning electron microscope (SEM) photograph which shows a cross-sectional view of a pellet of Example 1 according to the invention which shows the micropellets in the pellet structure.

FIG. 2 is a graph which shows the dissolution profile of pellets of oxybutynin in pH6.8 phosphate buffer which are prepared in Example 1 where the slow release profile is derived from the Example of the invention and the fast release profile is derived from the comparative Example.

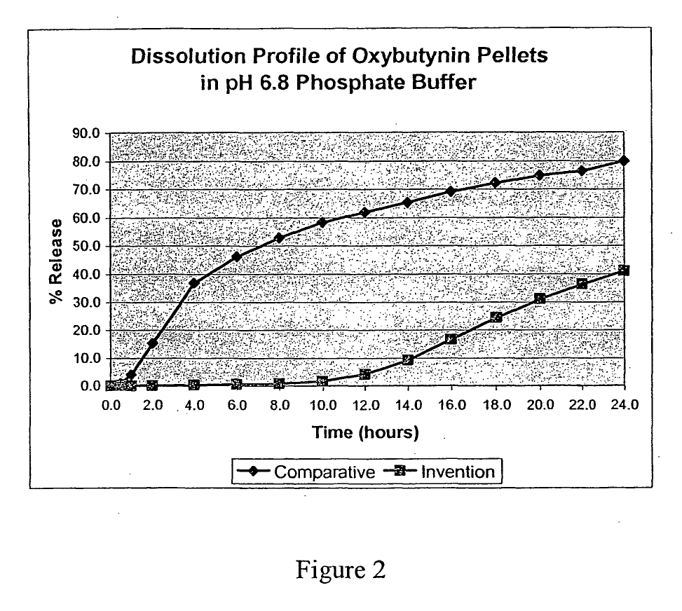

FIG. 3 is a scanning electron microscope (SEM) photograph of the comparative drug layered pellet of Example 1.

FIG. 4 is a scanning electron microscope (SEM) photograph of a cross-section of a pellet produced in Invention Test C which shows the agglomerated micropellet structure.

FIG. 5 is a scanning electron microscope (SEM) photograph of a cross-section of a pellet produced in Example 3 which shows the agglomerated micropellet structure.

FIG. 6 is a scanning electron microscope (SEM) photograph of a cross-section of a pellet produced in Example 4 which shows the agglomerated micropellet structure.

FIG. 7 is a diagram of a pellet having the micropellet structure which shows coated micropellets dispersed in a matrix with a first optional outer matrix layer and an second optional controlled release membrane.

### DETAILED DESCRIPTION OF THE INVENTION

The pellets of the invention are typically prepared using an apparatus which propels particles against a tangentially arranged inner wall in such a manner that a rolling motion is imparted to the moving pellets. A liquid is fed into an apparatus such as the apparatus disclosed in U.S. Pat. No. 6,449,689 which is adapted to allow for the introduction of powder during the operation of the apparatus. In one embodiment of the invention, the process of the invention involves the introduction of powder as a final step in the process in order to control and/or terminating pellet growth as well as assisting in the drying, rounding and smoothing of the pellets. The preferred apparatus is described in U.S. Pat. No. 6,449,869 and U.S. Pat. No. 6,354,728, both of which are incorporated by reference.

In one embodiment, the pellets of the invention, have a matrix which has a structure that results from the simultaneous application of a liquid stream containing a pharmaceutically acceptable diluent and a powder stream comprising a biologically active agent and a pharmaceutical excipient or a pharmaceutical excipient, alone, under drying conditions to form a pellet having a desired size. The liquid and powder stream components may be combined to form a single feed, if desired. At that point, an outer zone of the pellet may be formed by feeding dry powder to the tumbling bed of pellets in order to cause the pellets to grow to their selected final dimension as well as to dry and smooth the pellets into a highly uniform and highly spherical product.

When the biologically active material is a pharmaceutical, it may be any physiologically or pharmacologically active substance that produces a local or systemic effect, in animals, including warm-blooded mammals, humans and primates

The pharmaceutically acceptable liquid which is used in the formation of the pellets may comprise one or more components selected from the group consisting of biologically active ingredients, binders, diluents, disintegrants, lubricants, flavoring agents, coloring agents, surfactants, anti-sticking agents, osmotic agents, matrix forming polymers, film forming polymers, release controlling agents and mixtures thereof, in dissolved, suspended or dispersed form. Generally, only selected components will be employed to achieve the desired result for a given formulation. The particular formulation will determine if, when and how the listed components are added.

The active pharmaceutical that can be delivered includes inorganic and organic compounds without limitation, including drugs that act on the peripheral nerves, adrenergic receptors, cholinergic receptors, nervous system, skeletal muscles, cardiovascular system, smooth muscles, blood circulatory system, synaptic sites, neuroeffector junctional sites, endocrine system, hormone systems, immunological system, reproductive system, skeletal system, autocoid systems, alimentary and excretory systems, inhibitory of autocoid systems, alimentary and excretory systems, inhibitory of autocoids and histamine systems. The active drug that can be delivered for acting on these recipients include anticonvulsants, analgesics, anti-inflammatories, calcium antagonists, anesthetics, antimicrobials, antimalarials, antiparasitic, antihypertensives, antihistamines, antipyretics, alpha-adrenergic agonist, alpha-blockers, biocides, bactericides, bronchial dilators, beta-adrenergic blocking drugs, contraceptives, cardiovascular drugs, calcium channel inhibitors, depressants, diagnostics, diuretics, electrolytes, hypnotics, hormonals, hyperglycemics, muscle contractants, muscle relaxants, ophthalmics, psychic energizers, parasympathomimetics, sedatives, sympathomimetics, tranquilizers, urinary tract drugs, vaginal drugs, vitamins, nonsteroidal anti-inflammatory drugs, angiotensin converting enzymes, polypeptide drugs, and the like.

Exemplary drugs that are very soluble in water and can be delivered by the pellets of this invention include prochlorperazine, ferrous sulfate, aminocaproic acid, potassium chloride, mecamylamine hydrochloride, procainamide hydrochloride, amphetamine sulfate, amphetamine hydrochloride, isoproteronol sulfate, methamphetamine hydrochloride, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, cimetidine hydrochloride, theophylline cholinate, cephalexin hydrochloride, oxybutynin hydrochloride and the like.

Exemplary drugs that are poorly soluble in water and that can be delivered by the particles of this invention include diphenidol, meclizine hydrochloride, omeprazole prochlorperazine maleate, phenoxybenzamine, thiethylperzine maleate, anisindone, diphenadione, erythrityl tetranitrate, digoxin, isofluorophate, acetazolamide, methazolamide, bendro-flumethiazide, chlorpropamide, tolazamide, chlormadinone acetate, phenaglycodol, allopurinol, aluminum aspirin, methotrexate, acetyl sulfisoxazole, erythromycin, progestins, progestational, corticosteroids, hydrocortisone hydrocorticosterone acetate, cortisone acetate, triamcinolone, methyltestosterone, 17 beta-estradiol, ethinyl estradiol, ethinyl estradiol 3-methyl ether, prednisolone, 17 betahydroxyprogesterone acetate, 19 non-progesterone, norgesterel, norethindrone, norethisterone, norethiederone, progesterone, norgesterone, norethynodrel, and the like.

Examples of other drugs that can be formulated according to the present invention include aspirin, indomethacin, naproxen, fenoprofen, sulindac, indoprofen, nitroglycerin, isosorbide dinitrate, timolol, atenolol, alprenolol, cimetidine, clonidine, imipramine, levodopa, chloropromazine, methyldopa, dihydroxyphenylalamine, pivaloyloxyethyl ester of alpha-methyldopa hydrochloride, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, captopril, madol, propranolol hydrochloride, quanbenz, hydrochlorothiazide, ranitidine, flurbiprofen, fenbufen, fluprofen, tolmetin, alolofenac, mefanamic, flufenamic, difuninal, nimodipine, nitrendipine, nisoldipine, nicardipine, felodipine, lidoflazine, tiapamil, gallopamil, amlodipine, mioflazine, lisinolpril, enalapril, captopril, ramipril, endlapriate, famotidine, nizatidine, sucralfate, etintidine, tertatolol, minoxidil, chlordiazepoxide, chlordiazepoxide hydrochloride, diazepam, amitriptylin hydrochloride, impramine hydrochloride, imipramine pamoate, enitabas, buproprion, oxybutynin chloride and the like.

Other examples of biologically active materials include water soluble vitamins such as the B Vitamins, Vitamin C and the oil soluble vitamins such as Vitamin A, D, E and K. Neutraceuticals such as chondroitin, glucosamine, St. John's wort, saw palmetto and the like may also be formed into pellets according to the present invention.

In the case of pellets having a matrix and an outer layer, the matrix of the pellets may comprise, depending on the properties of the biological agent, from 0.1-90 wt % or from 3 to 80 wt % or from 5 to 60 wt % of a biologically active agent, based on the total weight of the pellet. Suitable binders for use in the formation of pellets include those materials that impart cohesive properties to the powdered biologically active material when admixed dry or in the presence of a suitable solvent or liquid diluent. These materials commonly include ethyl cellulose, hydroxypropyl methyl cellulose, propyl cellulose, starches such as pregelatinized starch, gelatin, methylcellulose, and acrylic copolymers such as Eudragit NE 30D; Eudragit RS 30D Eudragit RL30D, Eudragit S-100 and the like. Binders are used in an effective amount, e.g. 1 to 10 wt %, based on the total weight of liquid and binder to cause a sufficient degree of agglomeration of the powders that stable particles are rapidly formed.

An outer layer maybe formed by applying to the matrix pellet a powder which comprises a substantially dry, free flowing inert powder which is a pharmaceutical excipient which forms a non-tacky surface when placed in contact with water. Examples of such free flowing inert pharmaceutical excipient powders include water soluble and water insoluble materials. Examples of useful materials include microcrystalline cellulose, dicalcium phosphate, calcium sulfate, talc, an alkali metal stearate, silicon dioxide, sugars such as sucrose, dextrose, lactose, corn starch, calcium carbonate and the like which are used in a sufficient quantity to achieve the desired result. Osmotic agents, such as non-toxic inorganic salts, e.g. sodium chloride, potassium chloride, sodium dihydrogen phosphate and other materials which exert may also be added in amounts of 1-30%.

The powder which comprises a substantially dry, free flowing-inert pharmaceutical diluent powder, may also include an active biological agent. For example, a particle having an outer zone formed from a substantially dry, free flowing inert powder and a biological agent, may contain, depending on the properties of the biological agent, from 0.1-90 wt % or from 3 to 80 wt % or from 5 to 60 wt % of a biologically active agent, based on the total weight of the pellet.

Other additives that may be used in the pellet of the invention include diluents, lubricants, disintegrants, coloring agents and/or flavoring agents. The pellets will have a matrix which comprises a substantially uniform dispersion of pellets which are aggregated together. The micropellets may also comprise (a) a pharmaceutically active compound with or without an osmotic agent and/or a stabilizing agent and/or a pharmaceutical excipient or (b) a pharmaceutical excipient with or without an osmotic agent and/or a stabilizing agent. The matrix may comprise a) a pharmaceutically active compound with or without an osmotic agent and/or a stabilizing agent and/or a pharmaceutical excipient or (b) a pharmaceutical excipient with or without an osmotic agent and/or a stabilizing agent. Stabilizers will be selected to provide the necessary stabilizing environment required by the particular biologically active agent. In particular cases alkaline or acidic materials may be employed to modify the pH if necessary.

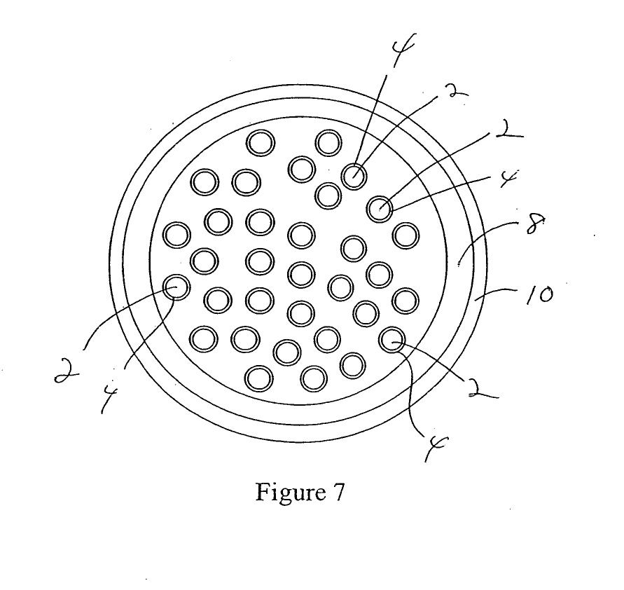

A water insoluble coating, as defined herein, is preferably placed around the micropellets. In selected situations, the pharmaceutically active compound and/or the pharmaceutical excipient may be sufficiently resistant to the action of water that it may be directly formed into micropellets that may be used in the invention without an additional water insoluble coating. FIG. 7 is a diagram of the pellet of the invention which shows the micropellets 2, that coated with a water insoluble coating 4, dispersed in matrix 6. A first optional outer matrix layer 8 may be built up on the pellet with or without a second optional controlled release membrane 10. For convenience only a representative number of the micropellets have been labeled with reference characters.

In conjunction with the pellets, a plurality of layers of biologically active materials, inert materials, or release controlling layers may be applied depending on the desired biological effect.

The pellets according to the invention may be made by using an apparatus that is described in U.S. Pat. No. 6,354,728. That apparatus comprises a rotor chamber having an axially extending cylindrical wall, means for passing air through said chamber from the bottom, spray means for feeding a liquid into said chamber, a rotor which rotates on a vertical rotor axis, said rotor being mounted in said rotor chamber, said rotor having a central horizontal surface and, in at least the radial outer third of said rotor, the shape of a conical shell with an outward and upward inclination of between 10° and 80°, said conical shell having a circularly shaped upper edge which lies in a plane which is perpendicular to the rotor axis, feed ports for introducing micropellets, a plurality of guide vanes having an outer end affixed statically to said cylindrical wall of said rotor chamber above a plane formed by the upper edge of said conical shell of said rotor and an inner end which extends into said rotor chamber and is affixed tangentially to said cylindrical wall of said rotor chamber and having, in cross-section to the rotor axis, essentially the shape of an arc of a circle or a spiral, such that said micropellets which are circulated by kinetic energy by said rotor under the influence of kinetic energy, moves from said rotor to an inside surface of said guide vanes before it falls back onto said rotor.

When the desired pellet size is substantially achieved the apparatus maybe allowed to run for a period of 3 to 15 minutes, and preferably 5 to 10 minutes to complete the smoothing of the pellets.

It is also contemplated that some additional drying at a temperature of from about 30 to 100° C., and preferably from about 40 to 90° C. until the moisture content is from 1 to 10 wt %, based on the total weight of the pellets depending on the particular biologically active material and/or the particular pharmaceutical excipients. Drying may be carried out in the preferred apparatus of the invention for making the pellets or in a separate dryer such as a fluid bed dryer.

The process is preferably based on the use of a minimal amount of liquid in order to avoid causing substantial swelling or gelation of any matrix forming materials which are placed on the pellet according to the invention.

The matrix forming material may be any swellable or non-swellable material that provides in vitro dissolution rates of a biologically active agent within the narrow ranges required to provide the desired plasma level of the biologically active agent over a desired interval which is typically 12 to 24 hours. Most matrix forming material will also provide for the release of the biologically active agent in a pH independent manner. Preferably the matrix is a controlled release matrix, although normal release matrices having a coating that controls the release of the drug may be used. Suitable water-swellable materials for inclusion in a controlled release matrix are

(a) Hydrophilic polymers, such as gums, cellulose ethers, acrylic resins and protein derived materials. Of these polymers, the cellulose ethers, especially hydroxyalkylcelluloses and carboxyalkylcelluloses, are preferred. The pellets may contain between 1% and 35 wt % of a hydrophilic or hydrophobic polymer.
(b) Digestible, long chain (C₈-C₅₀, especially C₁₂-C₄₀), substituted or unsubstituted hydrocarbons, such as fatty acids, fatty alcohols, glyceryl esters of fatty acids, mineral and vegetable oils and waxes. Hydrocarbons having a melting point of between 25° and 90° C. are preferred. Of these long chain hydrocarbon materials, fatty (aliphatic) alcohols are preferred. The pellets may contain up to 60% (by weight) of at least one digestible, long chain hydrocarbon.
(c) Polyalkylene glycols. The pellets may contain up to 60% (by weight) of at least one polyalkylene glycol.

One particular suitable matrix forming material comprises a water soluble hydroxyalkyl cellulose, at least one C₁₂-C₃₆, preferably C₁₄-C₂₂, aliphatic alcohol and, optionally, at least one polyalkylene glycol.

The hydroxyalkyl cellulose is preferably a hydroxy (C₁ to C₆) alkyl cellulose, such as hydroxypropylcellulose (HPC) or hydroxypropyl methylcellulose (HPMC). The nominal viscosity of the HPC or HPMC may be between 2,500 and 100,000 (2% w/v sol. at 20° C.) and preferably 5,000 to 50,000. The amount of the matrix forming material in the pellet will be determined, inter alia, by the precise rate of release required. This may be done by using conventional release rate testing procedures such as those described in U.S.P. 23, which testing procedures are incorporated by reference. When the pellets are formulated to contain a matrix polymer, the pellets will contain between 1% and 40 wt. %, especially between 5% and 20 wt. % of HPC or HPMC, based on the total weight of the pellets.

When forming pellets with water-swellable matrix forming materials, care should be exercised to prevent the matrix forming materials from swelling due to prolonged contact with liquid diluents in order to prevent the water-swellable matrix forming material from forming a gel during the pellet formation step.

Non-swellable matrix forming materials comprise water insoluble, dispersible polymers include the commercially available acrylic/methacrylic polymers as well as ethyl cellulose. The acrylic/methacrylic polymers are available under various tradenames such as Eudragit. These materials are used as non-swellable matrix forming polymers when they are admixed with biologically active compounds and various excipients which are formed into pellets according to the present invention. Generally from 1 to 30 wt %, of non-swellable matrix forming polymer, based on the weight of biologically active agent, excipient and non-swellable matrix forming polymer of may be admixed for the purpose of making a powder which may be formed into pellets according to the invention.

A release rate controlling polymer membrane may be applied to the pellets to provide for sustained release, delayed release, e.g. release in the small intestine by using a pH sensitive coating such as an enteric coating. Suitable enteric coatings include polymeric enteric coating material. The enteric coatings are “pH dependent” which describes the well known effect of an enteric coating which prevents release of the dosage form in the low pH conditions of the stomach but permits release in the higher pH conditions of the small intestine. The enteric coating will comprise from 1 to 25 wt % and preferably from 5 to 10 wt % of the total weight of the pellets. The enteric coating polymer may be selected from the group consisting of shellac, methacrylic acid copolymers, (Eudragit S or L) cellulose acetate phthalate, hydroxypropylmethylcellulose phthalate, hydroxypropylmethylcellulose acetate succinate, cellulose acetate trimellitate and polyvinyl acetate phthalate. Methacrylic acid copolymer, Type B USP/NFXXII which dissolves at a pH above about 6.0 is preferred. The thickness of the coating is selected to provide the desired release rate depending on the thickness of the coating and the particular coating.

A commercially available copolymer is Eudragit S100 which is based on methacrylic acid and methyl methacrylate and has a weight average molecular weight of about 150,000. Other auxiliary coating aids such as a minor amount (1-5 wt % based on the active core component and the total weight of the final coating) of a plasticizer such as acetyltributyl citrate, triacetin, acetylated monoglyceride, rape oil, olive oil, sesame oil, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, polyethyleneglycol (molecular weight of from 380 to 420), propylene glycol and mixtures thereof in combination with an antisticking agent which may be a silicate such as talc. An antisticking agent, such as talc may be added in an amount which is effective to prevent sticking of the pellets. These components may be added to the methacrylic acid copolymer in combination with appropriate solvents.

A sustained release coated pellet may be coated with a polymeric material which will substantially maintain its integrity in the varying pH conditions of the gastrointestinal tract but is permeable to the particular biologically active agent which is being formulated. The sustained release coating is used at a level that is selected to release the biologically active agent at a rate that will provide the desired in vivo release characteristics that will provide the desired plasma profile for the selected biologically active agent. Polymers such as ethyl cellulose, cellulose acetate, cellulose acetate butyrate, or an acrylic copolymer which when used in a sufficient amount will cause the coated pellet to release the biologically active agent after ingestion of the dosage form of the invention. Materials such as Eudragit RS 30D; RS 100; NE 30D; RL 30D or RL 100 may be used to prepare the delayed pulse pellet. One such useful material is an acrylate copolymer which has a permeability which is independent of pH. That acrylate copolymer is commercially available as Eudragit RS30D which is available as a 30 wt % aqueous dispersion of copolymers of acrylic and methacrylic acid esters, having a number average molecular weight of 150,000 with a low content of quaternary ammonium groups. Other auxiliary coating aids such as a minor amount (3-7 wt % based on the total weight of the active core component and the total weight of the final coating) of a plasticizer such as acetyltributyl citrate, triacetin, acetylated monoglyceride, rape oil, olive oil, sesame oil, acetyltriethylcitrate, glycerin sorbitol, diethyloxalate, diethylmalate, diethylfumarate, dibutylsuccinate, diethylmalonate, dioctylphthalate, dibutylsebacate, triethylcitrate, tributylcitrate, glyceroltributyrate, polyethyleneglycol (molecular weight of from 380 to 420), propylene glycol and mixtures thereof.

If a disintegrant is employed, it may comprise from 2 to 8 wt. % based on the total weight of the pellet, of starch, clay, celluloses, algins, gums and cross-linked polymers. Super disintegrants such as cross-linked cellulose, cross-linked polyvinylpyrrolidone, Croscarmellose sodium, carboxymethylcellulose and the like may also be employed if it desired to have a rapid release of the biologically active agent.

Conventional osmotic agents include non-toxic inorganic salts such as sodium chloride, potassium chloride, disodium phosphate and the like or water soluble non-toxic organic compounds such as lactose, sucrose, dextrose and the like. Antisticking agents such as talc may be employed to achieve any required result.

The pellets of the invention may be placed in hard or soft gelatin capsules to prepare finished dosage forms suitable for administration to a patient or they may be used to prepare compressed tablets using suitable cushioning agents, diluents, binders, disintegrants and lubricants.

### DESCRIPTION OF THE PREFERRED EMBODIMENTS

### Example 1

Micropellets of sodium chloride were made by the following procedure:

### Micropellet Composition:

| | |
|---|---|
| Sodium chloride | 3.0 Kg |
| Microcrystalline cellulose | 12.0 Kg |
| (Avicel PH101) | |

### Procedure:

1. Blend sodium chloride and microcrystalline cellulose in a vertical high shear granulator for 2 min.
2. Weigh 3 Kg of the blend for powder feeding portion
3. Spray 3.6 Kg of water at 500 g/min spray rate, atomization air pressure 2.0 bar.
4. Discharge the blend from high shear granulator, load the blend into an apparatus as described in U.S. Pat. No. 6,354,728.
5. Start the apparatus and spray water at 350 g/min. Process conditions follow:
   Inlet air temperature 17° C.
   Rotor speed 587 rpm, reduce to 350 rpm (5.5 m/sec.) after 1.6 Kg of water applied
   After 7.4 Kg of water applied, start powder feed at 330 g/min.
   Stop process after 8.8 Kg water is applied.
6. Discharge the wet micropellets from step 5. Dry in a fluid bed dryer. Final moisture 0.32%.

### Micropellet Particle Size Distribution

Determined using sieve analysis

| Sieve (#) | Size (micron) | % Retained |
|---|---|---|
| 30 | 600 | 4.2 |
| 35 | 500 | 4.0 |
| 40 | 425 | 8.5 |
| 45 | 355 | 23.6 |
| 50 | 300 | 49.4 |
| 60 | 250 | 10.0 |
| 80 | 180 | 0.2 |
| Pan | | 0.0 |

Bulk Density 0.9 g/cc

Coating of Micropellets with Ethyl Cellulose.

The micropellets prepared above were screened through sieves #40 and 60. Micropellets that retained on sieve #60 and passed through sieve #40 were coated using ethyl cellulose.

Coating Composition

| | |
|---|---|
| Ethyl cellulose | 0.11 Kg |
| Methanol | 2.09 Kg |
| Total solution | 2.20 Kg |

### Procedure:

1. Set up a fluid bed processor with 6″ Wurster column.
2. Prepare coating solution using composition specified.
3. Load 2.0 Kg of the salt micropellets (sieve cut #40/60) into the product container.
4. Start coating process. Process conditions follow:
   Inlet temperature 60° C.
   Atomization air pressure 2.5 bar.
   Partition height 20 mm.
   Air volume 7.2 g/min.
   Spray rate 7-18 g/min.
   Stop process after all coating solution applied.
   Total ethyl cellulose applied=110 g for 2.0 Kg of salt micropellets
   Quantity of ethyl cellulose=5.2% of coated pellets.

### Drug Layering

The ethyl cellulose coated micropellets were used as starting micropellets for the application of a drug layer in conjunction with the making of pellets containing the micropellets:

Drug layering composition

| | |
|---|---|
| Oxybutynin | 0.300 Kg |
| Stearic acid | 0.560 Kg |
| Microcrystalline cellulose | 0.140 Kg |
| Total powder blend | 1.000 Kg |

### Drug Layering Trial According to the Invention—HPMC (Methocel E5S)

(low radial velocity applied to the pellets)
Binder preparation

| | |
|---|---|
| HPMC - (Methocel E5) | 0.040 Kg |
| Purified water | 1.960 Kg |
| Starting pellets: ethyl cellulose coated salt micropellets | 1.0 Kg |

### Procedure:

1. Load 1.0 Kg ethyl cellulose coated salt micropellets (into the apparatus of U.S. Pat. No. 6,354,728)
2. Start apparatus according to U.S. Pat. No. 6,354,728. Start spraying HPMC solution. Process conditions follow:
   Inlet temperature 25° C.
   Initial rotor speed 500 rpm (7.9 m/sec.)
   Initial solution spray rate 30 g/min
   Process air volume 70 cubic meter/hour
3. When 142 g of HPMC solution is applied, the powder feed is started (drug layering composition) at 25 g/min.
4. After approx. 470 g of solution sprayed, rotor speed reduced to 400 rpm (6.3 m/sec.), powder feed rate reduced to 20 g/min and spray rate reduced to 25 g/min.
5. After 966 g solution sprayed, spray rated reduced to 20 g/min. After 1086 g solution sprayed, rotor speed increased to 50.0 rpm (7.9 m.sec.), then to 600 rpm (9.4 m/sec.). The rotor speed was then further increased to 800 (12.6 m/sec.), 1000 (15.7 m/sec.) and 1500 (23.6 m/sec.) rpm. The increase in rotor speed did not reduce the size of the pellets. The pellets from this particular experiment were (2-3 mm). Total power feed time 44 minutes.

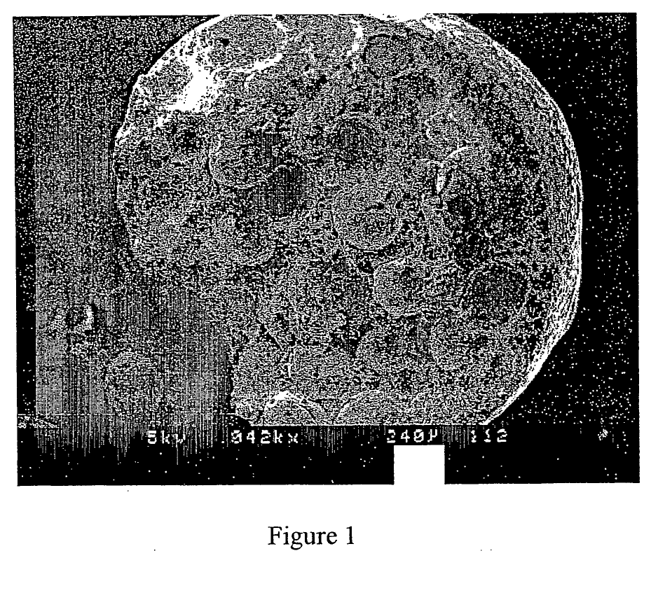

This procedure resulted in larger than expected pellets which when examined with a scanning electron microscope as shown in FIG. 1, have a structure where the micropellets were essentially agglomerated within a discrete pellet structure.

### Comparative Drug Layering Trial—PVP (Kollidon K90)

(High radial velocity applied to pellets)
Binder preparation

| | |
|---|---|
| PVP - (Kollidon K90) | 0.040 Kg |
| Purified water | 1.960 Kg |
| Starting pellets: ethyl cellulose coated salt micropellets | 1.0 Kg |

### Procedure:

1. Load 1.0 Kg ethyl cellulose coated salt micropellets (into the apparatus of U.S. Pat. No. 6,354,728)
2. Start apparatus according to U.S. Pat. No. 6,354,728. Start spraying PVC solution. Process conditions follow:
   Inlet temperature 25° C.
   Initial rotor speed 800 rpm (12.6 m/sec.)
   Initial solution spray rate 25 g/min
   Process air volume 70 cubic meter/hour
3. When 140 g of PVC solution is applied, the powder feed is started (drug layering composition) at 15 g/min.
4. After approx. 240 g of solution sprayed, powder feed rate increased to 25 g/min.
5. After approx. 290 g of solution sprayed, spray rate reduced to 20 g/min. After 363 g solution sprayed, spray rate increased to 25 g/min.
6. After 695 g solution sprayed and approx. 22 minutes after starting powder feed, addition of powder was stopped due to loss of air volume control in the apparatus. Strong suction from the apparatus insert resulted in a large quantity of powder being inadvertently fed to the batch. After 1014 g of solution was sprayed, the process was terminated. (Total time of power feed 25 minutes)

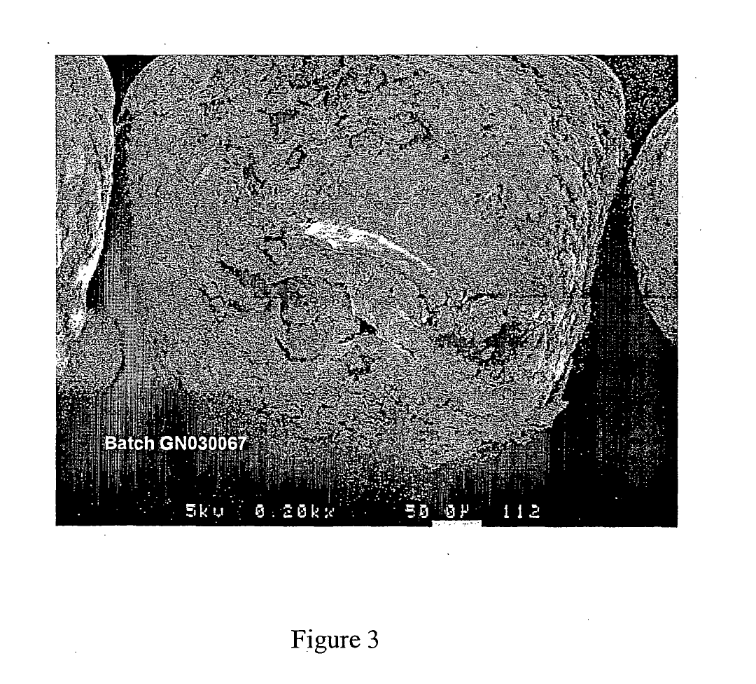

The pellets produced in this trial were individual pellets with a layer of drug around the core pellets (See FIG. 3

### Sustained Release Coating of Drug Pellets

The oxybutynin pellets prepared as described above, were subsequently coated using the same coating formulation (see below). Both batches were coated to 9% coating level. In-process samples were taken at 3, 5 and 8% coating levels. The purpose is to compare dissolution profile of these two pellet batches.

### Sustained Release Coating of the First Batch Oxybutynin Pellets:

A first batch of oxybutynin pellets prepared as described above, are screened using sieves no. 20 and 40. The fraction of the pellets that passed through sieve no. 20 and were retained on sieve no. 40 (425-850 micron) and are coated with a polymer for sustained release.

Coating solution

| | |
|---|---|
| Methanol | 1.566 Kg |
| HPMC - (Methocel E5) | 0.010 Kg |
| Ethocel (Std 10 Premium) | 0.090 Kg |
| Starting pellets: Oxybutynin chloride core pellet prepared as | 1.0 Kg |
| described above: | |

### Procedure:

1. Load Oxybutynin chloride pellet batch into a 6″ Wurster in a GPCG-1 (Glatt GmbH)
2. Start the process. Start spraying the coating solution. Process conditions follow:
   Inlet temperature 50° C.
   Solution spray rate log/min (range approx. 8-12 g/min)
   Process air volume approx 7 meter/sec
3. When 1648.4 g of coating solution applied, stop spraying.
4. Dry the pellets for 4 minutes.

### Sustained Release Coating of Second Batch of Oxybutynin Chloride Pellets (Control)

The pellet batch was screened using sieves no. 8 and 12. The fraction of pellets that passed through sieve no. 8 and were retained on sieve no. 12 (1.70-2.36 mm) were coated using the same coating formulation as used for the first coating batch, to the same level, for sustained release.

Coating solution

| | |
|---|---|
| Methanol | 1.566 Kg |
| HPMC - (Methocel E5) | 0.010 Kg |
| Ethocel (Std 10 Premium) | 0.090 Kg |
| Starting pellets: Oxybutynin chloride core pellet | 1.0 Kg |

### Procedure:

1. Load Oxybutynin chloride core pellets into 6″ Wurster, GPCG-1 (Glatt GmbH)
2. Start process. Start spraying coating solution. Process conditions similar to the first batch.
3. When 1648.4 g of coating solution applied, stop spraying.
4. Dry the pellets for 4 minutes.

### Dissolution Profiles of the Coated Pellets

Dissolution Results of Oxybutynin Cl Pellet Batches

| | Comparative | Invention |
|---|---|---|
| Time (hour) | % Released | % Released |
| 0.0 | 0.0 | 0.0 |
| 0.5 | 0.9 | 0.1 |
| 1.0 | 4.0 | 0.0 |
| 2.0 | 15.2 | 0.0 |
| 4.0 | 36.8 | 0.2 |
| 6.0 | 46.1 | 0.5 |
| 8.0 | 52.8 | 0.7 |
| 10.0 | 58.3 | 1.6 |
| 12.0 | 61.8 | 4.1 |
| 14.0 | 65.3 | 9.1 |
| 16.0 | 69.1 | 16.5 |
| 18.0 | 72.2 | 24.3 |
| 20.0 | 74.8 | 31.0 |
| 22.0 | 76.4 | 36.2 |
| 24.0 | 79.9 | 40.9 |

### Discussion:

The coated pellet example of the invention (micropellets within pellets) showed different dissolution characteristics when compared to the second coated micropellet batch (comparative) at the same coating level (8%) using a water insoluble polymer (ethyl cellulose in methanol). The second pellet batch shows a first order release typical of pellets that are coated using a water insoluble polymer such as ethyl cellulose.

For the invention, there was no drug release in the first two hours and very slow release up to 8 hours (less than 1% released) as compared to the comparative which released 15.2% of drug in the first two hours. This behavior was followed by a rapid increase in release, a desired characteristic of a pulsatile drug delivery system. By varying the makeup of the pellets and the composition and amount of coating material, it is possible to adjust the dissolution profile of active pharmaceuticals to obtain the desired drug release characteristics.

In the course of two drug layering experiments, one produced a micropellet in a pellet structure which was not expected as the product of a drug layering procedure. The other experiment produced individual drug pellets, typical of what would be expected from a drug layering procedure. Subsequent coating of these drug pellets with the same controlled/modified release membrane produces finished pellets with different dissolution profiles.

### Example 2

These experiments demonstrate that micropellets of sodium chloride coated with ethyl cellulose will aggregate into a pellet containing micropellets provides the micropellets are propelled at the proper rate of speed and the proper spray rate and the powder feed is maintained.

### Comparative Experiment A:

Micropellets of uncoated 20% sodium chloride/80% microcrystalline cellulose, prepared as described in Example 1 were loaded into the apparatus described in U.S. Pat. No. 6,354,728 and the apparatus was started and the following process conditions were used:
Inlet temperature 25° C.
Initial rotor speed 500 rpm (70.9 m/sec.)
Initial solution spray rate 25 g/min
Process air volume 70 cubic meter/hour

The binder that is sprayed is a 2% w/w solution of low viscosity hydroxypropyl methyl celluose (HPMC) (Methocel E-5). When 312 g of the HPMC solution is sprayed, the powder feed of 1.0 Kg. of microcrystalline cellulose (Avicel PH101) is started at 25 g/min and the rotor speed is reduced to 400 rpm (6.3 m/sec). After 1200 g of solution was sprayed, the powder feed was finished. The total elapsed time is about 39 minutes.

Results: The pellets grew in size with some agglomeration but no pellets were formed that had micropellets in a larger pellet. The micropellet in a pellet structure was not found because the starting micropellets were not coated.

### Comparative Experiment B:

The procedure of Comparative Experiment A was repeated using coated 20% sodium chloride-80% microcrystalline cellulose pellets that were coated with 0.11 Kg of ethyl cellulose in 2.09 methanol as described in Example 1. The micropellets were loaded into the apparatus used in Comparative Experiment A and process conditions similar to those used in Comparative Experiment A and the same solution was sprayed and the same powder feed was used. The procedure varied in that after 250 g of the HPMC solution was sprayed, the HPMC solution feed rate was increased to 30 g/min and the powder feed rate was started at 20 g/min. After about 1500 g of HPMC-solution was sprayed, the powder feed was finished over a total of about 43 minutes.

Results: The pellets grew in size and had some powder adhering to the sides. A few agglomerates were formed but the finished pellets were not micropellets in a larger pellet. The reason why the micropellet in a pellet structure was not formed is that the spray rate was too low.

### Invention Experiment C

Comparative Test B was substantially repeated except that the initial solution spray rate was 60 g/min instead of 25 g/min. When about 280 g of the HPMC solution was applied, the solution spray rate was reduced to 45 g/min. When 305 g of HPMC solution was applied, the powder feed was started at 20 g/min. and increased to 30 g/min. During powder feed the rotor speed was between 400 rpm (6.3 m/sec.) and 500 rpm (7.9 m.sec.) and the spray rate was varied between 25 to 65 g/min. After 1482 g of solution was sprayed, the powder feeding was finished. The cycle time was about 28 minutes.

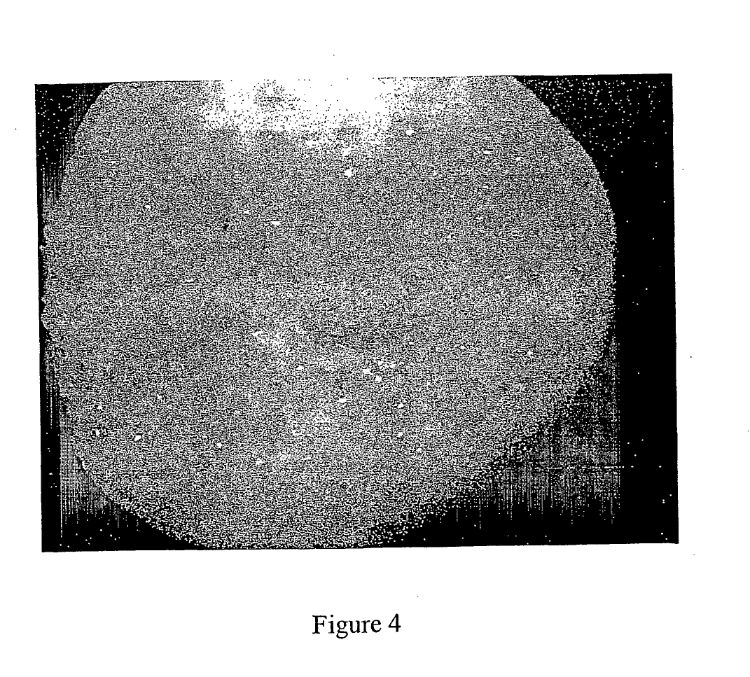

Results: The finished pellets were not uniform is size but they were micropellets agglomerated into a pellet. FIG. 4 is a cross-section of a pellet produced in Invention Experiment C which shows the micropellets agglomerated into a larger pellet.

### Example 3

This Example demonstrates that the micropellet in a pellet structure will form if polyvinylpyrrolidone is used as a binder in place of HPMC.

Micropellets of coated 20% sodium chloride/80% microcrystalline cellulose, prepared as described in Example 1 were loaded into the apparatus described in U.S. Pat. No. 6,354,728 and the apparatus was started and the following process conditions were used:
Inlet temperature 25° C.
Initial rotor speed 450 rpm (7.9 m/sec.)
Initial solution spray rate 60 g/min
Process air volume 70 cubic meter/hour

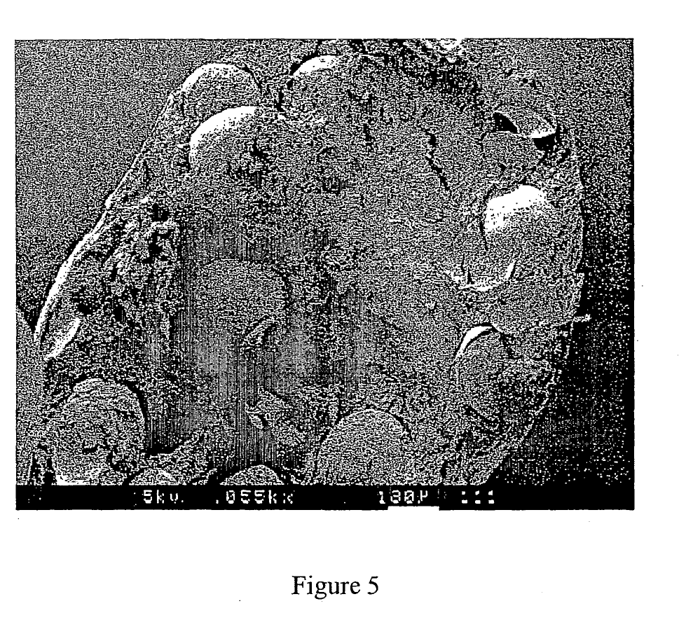

The binder that is sprayed is a 2% w/w solution of polyvinylpyrrolidone (PVP) (Kollidon K90). When 145 g of the PVP solution is sprayed, the powder feed of 1.0 Kg. of microcrystalline cellulose (Avicel PH101) is started at 30 g/min and the spray rate was reduced to 45 g/min. During powder feed the spray rate was varied between 45-65 g/min. and the rotor speed was kept constant at 400 rpm (7.9 m/sec.). After 1560 g of solution was sprayed, the powder feed was finished. A cross-section of an SEM of a pellet of Example 3 is shown in FIG. 5 where the structure of the micropellets in a pellet is clearly shown.

### Example 4

This Example shows that the micropellets will agglomerate to form a pellet even if no binder is used in the spraying media during the agglomeration procedure.

Micropellets of 20% sodium chloride-80% microcrystalline cellulose pellets that were coated as described in Example 1 were loaded into the apparatus described in U.S. Pat. No. 6,354,728 and the apparatus was started and the following process conditions were used:
Inlet temperature 25° C.
Initial rotor speed 300 rpm (4.7 m/sec.)
Initial solution spray rate 60 g/min
Process air volume 70 cubic meter/hour

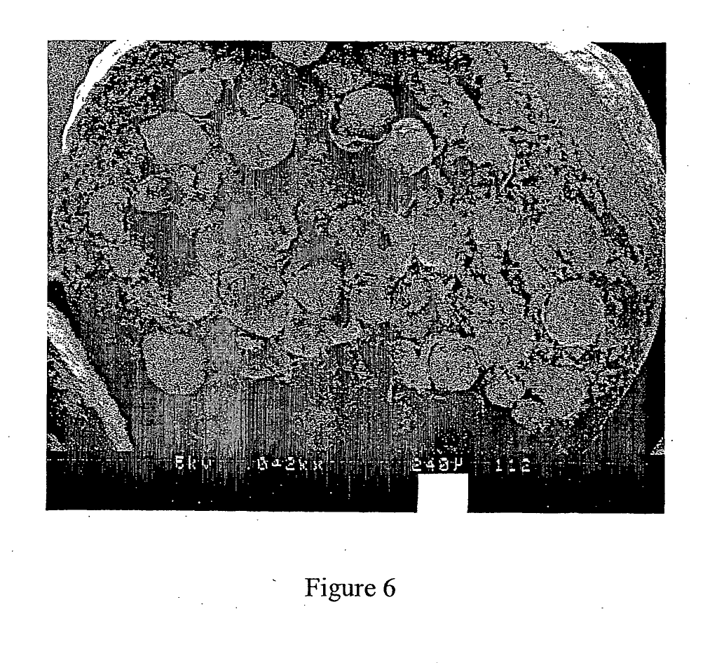

Water is sprayed and when 145 g of water is applied, the powder feed is started at 30 g/min. During powder feed, the spray rate was adjusted between 25 and 60 g/min with the rotor speed at 300 rpm (4.7 m/sec.). When the powder feed ended, 934 g. of water had been sprayed. A cross-section of an SEM of a pellet of Example 4 is shown in FIG. 6 where the structure of the micropellets in a pellet is clearly shown.

1. A pellet which is adapted for use in the delivery of a biologically active agent, said pellet comprising a plurality of micropellets which are bound together to form a pellet.

2. A pellet as defined in claim 1 wherein said micropellets comprise a biologically active agent and a binder.

3. A pellet as defined in claim 1 wherein said micropellets comprise a osmotic agent and a pharmaceutical excipient.

4. A pellet as defined in claim 1 wherein said micropellets comprise a biologically active agent and a pharmaceutical excipient.

5. A pellet as defined in claim 1 wherein said micropellets comprise a biologically active agent, a pharmaceutical excipient and a stabilizer.

6. A pellet as defined in claim 1 wherein said pharmaceutical excipient is selected from the group consisting of microcrystalline cellulose, dicalcium phosphate, calcium sulfate, talc, an alkali metal stearate, silicon dioxide and calcium carbonate.

7. A pellet as defined in claim 1 wherein the micropellets comprise from 0.1-95 wt % of one or more pharmaceutically acceptable binders and or excipients and 99.9-5.0 wt % of a biologically active agent.

8. A pellet as defined in claim 1 wherein the pharmaceutical excipient comprises from 0.1-99 wt % of a biologically active agent.

9. A pellet as defined in claim 1 wherein said pharmaceutical excipient comprises microcrystalline cellulose and from 0.1-99 wt % of a biologically active agent.

10. A pellet as defined in claim 1 wherein said micropellets comprise one or more components selected from the group consisting of lubricants, disintegrants, flavors, surfactants, stabilizers, anti-sticking agents, osmotic agents and mixtures thereof.

11. A pellet as defined in claim 1 wherein said pharmaceutical excipient additionally comprises one or more components selected from the group consisting of binders, diluents, disintegrants, lubricants, flavors, surfactants, anti-sticking agents, osmotic agents, stabilizers, and mixtures thereof.

12. A pellet as defined in any one of claim 1 wherein said micropellets or said pharmaceutical excipient comprises a swellable matrix forming polymer.

13. A pellet as defined in claim 1 wherein said micropellet or said pharmaceutical excipient comprises a non-swellable matrix forming polymer.

14. A pellet as defined in any one of claim 1 wherein said pellet is provided with an outer layer comprising a swellable matrix forming polymer and a non-swellable matrix forming polymer.

15. A pellet as defined in any one of claim 1, having an outer layer or layers which comprise a release rate controlling polymer.

16. A pellet as defined in any claim 10 wherein said swellable polymer is selected from the group consisting of hydroxypropyl methyl cellulose, hydroxypropyl cellulose, hydroxyethyl cellulose and carboxypolymethylene.

17. A pellet as defined in any claim 13 wherein said release rate controlling polymers are selected from the group consisting of ethyl cellulose, methacrylic acid copolymers, cellulose acetate phthalate, hydroxypropylmethylcellulose phthalate, hydroxypropylmethylcellulose acetate succinate, cellulose acetate trimellitate and polyvinyl acetate phthalate.

18. A process for making pharmaceutical pellets as defined in claim 1 wherein micropellets are contacted with a pharmaceutically acceptable liquid or a solution or dispersion of a binder as said micropellets are subjected to a rolling movement, and
(b) feeding a sufficient amount of a substantially dry, pharmaceutical excipient in the form of a free flowing powder which forms a non-tacky surface when placed in contact with water to provide on said pellets an outer zone having an external arcuate surface.

19. A process for making solid pellets which comprise micropellets which includes a biologically active agent, said process comprising:
(a) feeding micropellets to an operating apparatus which comprises a rotor chamber having an axially extending cylindrical wall, means for passing air through said chamber from the bottom, spray means for feeding a liquid into said chamber, a rotor which rotates on a vertical rotor axis, said rotor being mounted in said rotor chamber, said rotor having a central horizontal surface and, in at least the radial outer third of said rotor, the shape of a conical shell with an outward and upward inclination of between 10° and 80°, said conical shell having a circularly shaped upper edge which lies in a plane which is perpendicular to the rotor axis, feed ports for introducing said powdered excipient, a plurality of guide vanes having an outer end affixed statically to said cylindrical wall of said rotor chamber above a plane formed by the upper edge of said conical shell of said rotor and an inner end which extends into said rotor chamber and is affixed tangentially to said cylindrical wall of said rotor chamber and having, in cross-section to the rotor axis, essentially the shape of an arc of a circle or a spiral, such that said powdered product which is circulated by kinetic energy by said rotor under the influence of kinetic energy, moves from said rotor to an inside surface of said guide vanes before falling back onto said rotor;
(b) rotating said rotor, while feeding air and spraying a solution or a dispersion of a pharmaceutically acceptable liquid with or without a binder into said rotor chamber for a sufficient amount of time to form pellets having a desired diameter; and
(c) feeding a sufficient amount of a dry solid, pharmaceutical excipient to provide on said particles an outer zone comprising a layer formed from said substantially dry, free flowing inert powder.

20. A process as defined in claim 19 wherein said micropellets in step (a) comprise a biologically active agent and said dry solid, pharmaceutical excipient is selected from the group consisting of microcrystalline cellulose, dicalcium phosphate, calcium sulfate, talc, an alkali metal stearate, silicon dioxide, calcium carbonate and mixtures thereof.

21. A process as defined in claim 19 wherein the powder mixture in step (a) comprises a biologically active agent and an inert powder that is microcrystalline cellulose.

22. A process as defined in claim 19 wherein the biologically active compound is selected from the group consisting of vitamins, nutrients, pharmaceuticals and mixtures thereof.

23. A process as defined in claim 19 wherein the biologically active agent is a pharmaceutically active compound.

24. A process as defined in claim 19 wherein the binder is selected from the group consisting of hydroxy propyl cellulose, hydroxypropyl methyl cellulose, polyvinyl pyrrolidone and copolymers of polyvinyl pyrrolidone and vinyl acetate.

25. A process for making discrete substantially spherical pellets which comprise micropellets, said process comprising:
(a) feeding, micropellets which comprise a biologically active agent and a binder, said micropellets being pre-wetted with from 5-60% of a pharmaceutically acceptable liquid diluent, based on the total weight of the micropellets and the liquid diluent, to an operating apparatus which comprises a rotor chamber having an axially extending cylindrical wall, means for passing air through said chamber from the bottom, spray means for feeding a liquid into said chamber, a rotor which rotates on a vertical rotor axis, said rotor being mounted in said rotor chamber, said rotor having a central horizontal surface and, in at least the radial outer third of said rotor, the shape of a conical shell with an outward and upward inclination of between 10° and 80°, said conical shell having a circularly shaped upper edge which lies in a plane which is perpendicular to the rotor axis, feed ports for introducing said powdered excipient, a plurality of guide vanes having an outer end affixed statically to said cylindrical wall of said rotor chamber above a plane formed by the upper edge of said conical shell of said rotor and an inner end which extends into said rotor chamber and is affixed tangentially to said cylindrical wall of said rotor chamber and having, in cross-section to the rotor axis, essentially the shape of an arc of a circle or a spiral, such that said powdered product which is circulated by kinetic energy by said rotor under the influence of kinetic energy, moves from said rotor to an inside surface of said guide vanes before falling back onto said rotor; and
(b) rotating said rotor, while feeding air and spraying a pharmaceutically acceptable binder into said rotor chamber for a sufficient amount of time to form pellets having micropellets and (c) feeding a sufficient amount of a dry, solid, pharmaceutical excipient which comprises a biologically active agent and a binder or a free flowing inert powder which forms a non-tacky surface in contact with water to form said outer zone on said pellets.

26. A process as defined in claim 25 wherein in step (c) dry solid, pharmaceutical diluent in an amount that is equivalent to 5 to 35 wt. % of the micropellets that were initially fed to the apparatus, is added and the apparatus is allowed to run for a period of time to form said outer zone.

27. A process as defined in claim 25 wherein said powder which comprising a biologically active agent includes microcrystalline cellulose and optionally comprises one or more components selected from the group consisting of binders, diluents, lubricants, disintegrants, flavors, surfactants, anti-sticking agents, osmotic agents and mixtures thereof.

28. A process as defined in claim 25 wherein the biologically active compound is selected from the group consisting of vitamins, nutrients, pharmaceuticals and mixtures thereof.

29. A process as defined in claim 25 wherein the biologically active agent is a pharmaceutically active compound.

30. A process as defined in claim 25 wherein the pharmaceutically acceptable liquid diluent is water.

31. A pharmaceutical dosage form which comprises coated pellets having as a core a pellet as defined in claim 1 and one or more release rate controlling coatings selected from the group consisting of delayed release coatings and sustained release coatings or mixtures thereof.

32. A pharmaceutical dosage form as defined in claim 31 wherein the controlled release coating is a sustained release coating.

33. A pharmaceutical dosage form as defined in claim 31 wherein the controlled release coating is a delayed release coating.

33. A pharmaceutical dosage form as defined in claim 30 wherein the dosage form includes different populations of coated pellets having different controlled release coatings.

34. A pharmaceutical dosage form as defined in claim 31 wherein the dosage form is a hard gelatin capsule.
